SEQUENCE LISTING (19 sequences)

SEQ ID NO: 1 - DNA, 32 bp, Artificial Sequence
  synthetic PCR amplification primer Rep1-U for plasmid pUC18-RYUR construction
gggcccggatccgagcagcataaacgactgct

SEQ ID NO: 2 - DNA, 32 bp, Artificial Sequence
  synthetic PCR amplification primer Rep1-D for plasmid pUC18-RYUR construction
gggccctctagaacgctcaatgttgttcatga

SEQ ID NO: 3 - DNA, 32 bp, Artificial Sequence
  synthetic PCR amplification primer Rep2-U for plasmid pUC18-RYUR construction
gggcccgtcgacgagcagcataaacgactgct

SEQ ID NO: 4 - DNA, 32 bp, Artificial Sequence
  synthetic PCR amplification primer Rep2-D for plasmid pUC18-RYUR construction
gggcccctgcagacgctcaatgttgttcatga

SEQ ID NO: 5 - DNA, 32 bp, Artificial Sequence
  synthetic PCR amplification primer URA3-U for plasmid pUC18-RYUR construction
gggccctctagagtagtctagtacctcctgtg

SEQ ID NO: 6 - DNA, 32 bp, Artificial Sequence
  synthetic PCR amplification primer URA3-D for plasmid pUC18-RYUR construction
gggcccgtcgacgaaaagtgccacctgacgtc

SEQ ID NO: 7 - DNA, 32 bp, Artificial Sequence
  synthetic PCR amplification primer GLT1 prom-U for plasmid YIplac211-P-PGK1-GLT1 construction
gggcccggtacctttctgagcactgtcaggag

SEQ ID NO: 8 - DNA, 32 bp, Artificial Sequence
  synthetic PCR amplification primer GLT1 prom-D for plasmid YIplac211-P-PGK1-GLT1 construction
gggcccggatcctgatttcaacactggcatgc

SEQ ID NO: 9 - DNA, 32 bp, Artificial Sequence
  synthetic PCR amplification primer GLT1-U for plasmid YIplac211-P-PGK1-GLT1 construction
gggcccgtcgacatgccagtgttgaaatcaga

SEQ ID NO: 10 - DNA, 32 bp, Artificial Sequence
  synthetic PCR amplification primer GLT1-D for plasmid YIplac211-P-PGK1-GLT1 construction
gggcccctgcagttttagtatcgaccatttca

SEQ ID NO: 11 - DNA, 33 bp, Artificial Sequence
  synthetic PCR amplification primer PGK1 prom-U for plasmid YIplac211-P-PGK1-GLT1 construction
gggcccggatccaggcatttgcaagaattactc

SEQ ID NO: 12 - DNA, 38 bp, Artificial Sequence
  synthetic PCR amplification primer PGK1 prom-D for plasmid YIplac211-P-PGK1-GLT1 construction
gggcccgtcgactgttttatatttgttgtaaaaagtag

SEQ ID NO: 13 - DNA, 59 bp, Artificial Sequence
  synthetic PCR amplification primer KGPD1-U for GPD1 deletion
cacattccaaaggatttcagaggcgagggcaaggacgtcgacgacgttgtaaaacgacg

SEQ ID NO: 14 - DNA, 59 bp, Artificial Sequence
  synthetic PCR amplification primer KGPD1-D for GPD1 deletion
agtgggggaaagtatgatatgttatctttctccaataaatggaaacagctatgaccatg

SEQ ID NO: 15 - DNA, 59 bp, Artificial Sequence
  synthetic PCR amplification primer KGPD2-U for GPD2 deletion
ctctttccctttccttttccttcgctccccttccttatcaacgacgttgtaaaacgacg

SEQ ID NO: 16 - DNA, 59 bp, Artificial Sequence
  synthetic PCR amplification primer KGPD2-D for GPD2 deletion
gcaacaggaaagatcagagggggagggggggggagagtgtggaaacagctatgaccatg

SEQ ID NO: 17 - DNA, 59 bp, Artificial Sequence
  synthetic PCR amplification primer KFPS1-U for FPS1 deletion
tcaacaaagtataacgcctattgtcccaataagcgtcggtacgacgttgtaaaacgacg

SEQ ID NO: 18 - DNA, 59 bp, Artificial Sequence
  synthetic PCR amplification primer KFPS1-D for FPS1 deletion
catcatgtatagtaggtgaccaggctgagttcatgtcaacggaaacagctatgaccatg

SEQ ID NO: 19 - protein, 6 aa, Artificial Sequence
  synthetic 6xHis tag purification sequence
HHHHHH

(19) United States
(12) Patent Application Publication
(10) Pub. No.: US 2010/0291652 A1
(43) Pub. Date: Nov. 18, 2010

(54) YEAST STRAINS AND METHODS OF MAKING AND USING SUCH YEAST STRAINS

(75) Inventor: Pingsheng Ma, Tianjin (CN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Tianjin University

(21) Appl. No.: 12/294,851

(22) PCT Filed: Aug. 6, 2008
(86) PCT No.: PCT/CN08/71884

§ 371 Date: Aug. 2, 2010

(51) Int. Cl. C12P 1/02; C12N 1/19; C12N 15/74

(52) U.S. Cl. 435/171; 435/254.2; 435/254.21; 435/471

(57) ABSTRACT

The present disclosure provides genetically-modified yeast that are able to produce more ethanol and less glycerol than yeast lacking the corresponding genetic modifications. The approaches described herein involve disrupting the ability of the yeast to produce and/or transport glycerol and increasing the amount of a polypeptide involved in maintaining the redox balance of the yeast cell.

### TECHNICAL FIELD

This document relates to genetically-engineered yeast.

### BACKGROUND

Ethanol, which is most commonly produced by anaerobic fermentations with S. cerevisiae, is one of the most important products originating from the biotechnological industry with respect to both value and amount. However, the bioethanol business is operating on tight profit margins, and formation of glycerol, the major by-product of bioethanol production, consumes up to eight percent of the carbon sources in industrial ethanol fermentations. Therefore, elimination or reduction of glycerol formation to optimize the ethanol yield in order to ensure an efficient utilization of the carbon sources is of great importance for bioethanol industry's long-term economic viability.

### SUMMARY

The present disclosure describes genetic modifications in yeast that disrupt the ability of yeast to produce glycerol. Yeast that have been genetically modified as described herein typically produce decreased amounts of glycerol and increased amounts of ethanol compared to yeast that lacks the corresponding genetic modifications.

In one aspect, yeast that include a first genetic modification, a second genetic modification, and a third genetic modification are provided. In one embodiment, the first genetic modification disrupts a polypeptide involved in the synthesis of glycerol; the second genetic modification disrupts a polypeptide that transports or helps transport glycerol out of the cell; and the third genetic modification increases the amount of a polypeptide that maintains the redox balance in the cell. In another embodiment, the first genetic modification reduces expression of a nucleic acid encoding a GPDH polypeptide, essentially eliminates expression of a nucleic acid encoding a GPDH polypeptide, or results in an absence of a functional GPDH polypeptide, thereby disrupting glycerol synthesis and resulting in an accumulation of one or more precursors of glycerol; the second genetic modification reduces expression of a nucleic acid encoding a glycerol channel polypeptide, essentially eliminates expression of a nucleic acid encoding a glycerol channel polypeptide, or results in an absence of a functional glycerol channel polypeptide, thereby resulting in an accumulation of glycerol in the yeast; and the third genetic modification increases the amount of a polypeptide that reoxidizes NADH.

In another aspect, a S. cerevisiae yeast comprising a first genetic modification, a second genetic modification, and a third genetic modification is provided. In this embodiment, the first genetic modification reduces expression of a nucleic acid encoding a Gpd1p or Gpd2p polypeptide, essentially eliminates expression of a nucleic acid encoding a Gpd1p or Gpd2p polypeptide, or results in an absence of a functional Gpd1p or Gpd2p polypeptide; the second genetic modification reduces expression of a nucleic acid encoding a Fps1p polypeptide, essentially eliminates expression of a nucleic acid encoding a Fps1p polypeptide, or results in an absence of a functional Fps1p polypeptide; and the third genetic modification results in an increase in the amount of glutamate synthase polypeptide or an increase in the activity of a glutamate synthase polypeptide.

The first or second genetic modification can be a genetically-engineered point mutation, deletion, or insertion. In certain embodiments, the first or second genetic modification reduces expression of the polypeptide by at least 30%. The third genetic modification can be the presence of a strong promoter operably linked to a nucleic acid encoding the polypeptide. In addition to a first, second and third genetic modification, yeast further can include one or more additional genetic modifications.

The yeast described herein produce reduced amounts of glycerol and increased amounts of ethanol compared to yeast lacking a corresponding first, second and/or third genetic modification. In certain instances, yeast described herein can produce up to about 3% more ethanol than yeast lacking a corresponding first, second and/or third genetic modification. The yeast disclosed herein can be S. cerevisiae. The yeast disclosed herein can be used in methods of fermenting a biomass. Such methods include contacting biomass with yeast genetically engineered as described herein.

In still another aspect, methods of making (e.g., genetically engineering) yeast are provided. Such methods typically include introducing a first genetic modification into the yeast, wherein the first genetic modification is in a nucleic acid that encodes a polypeptide involved in the synthesis of glycerol; introducing a second genetic modification into the yeast, wherein the second genetic modification is in a nucleic acid that encodes a polypeptide that transports or helps transport glycerol out of the cell; and introducing a third genetic modification into the yeast, wherein the third genetic modification increases the amount of a polypeptide that maintains the redox balance of the yeast cells. In one embodiment, the first genetic modification is in a nucleic acid that encodes a GPDH polypeptide, the second genetic modification is in a nucleic acid that encodes a glycerol channel polypeptide, and the third genetic modification results in over-expression of a polypeptide that reoxidizes NADH. Yeast produced by such methods typically produce less glycerol and more ethanol than a corresponding yeast lacking the first, second and third genetic modifications.

In one embodiment, yeast are provided that includes a first genetic modification, a second genetic modification, and a third genetic modification. In this embodiment, the first genetic modification essentially eliminates expression of a nucleic acid encoding a Gpd2p polypeptide; the second genetic modification essentially eliminates expression of a nucleic acid encoding a Fps1p polypeptide; and the third genetic modification results in an increase in the amount of a glutamate synthase polypeptide. FTG2 is a representative yeast strain according to this embodiment.

In one embodiment, yeast are provided that include a first genetic modification and a second genetic modification. In this embodiment, the first genetic modification reduces expression of a nucleic acid encoding a Fps1p polypeptide, essentially eliminates expression of a Fps1p polypeptide, or results in an absence of a functional Fps1p polypeptide; and the second genetic modification results in an increase in the amount of a glutamate synthase polypeptide or an increase in the activity of a glutamate synthase polypeptide.

In one embodiment, yeast are provided that include a first genetic modification and a second genetic modification. In this embodiment, the first genetic modification reduces expression of a nucleic acid encoding a Gpd1p polypeptide, essentially eliminates expression of a nucleic acid encoding a Gpd1p polypeptide, or results in an absence of a functional Gpd1p polypeptide; and the second genetic modification results in an increase in the amount of a glutamate synthase polypeptide or an increase in the activity of a glutamate synthase polypeptide.

In one embodiment, yeast are provided that include a first genetic modification and a second genetic modification. In this embodiment, the first genetic modification reduces expression of a nucleic acid encoding a Gpd2p polypeptide, essentially eliminates expression of a nucleic acid encoding a Gpd2p polypeptide, or results in an absence of a functional Gpd2p polypeptide; and the second genetic modification results in an increase in the amount of a glutamate synthase polypeptide or an increase in the activity of a glutamate synthase polypeptide.

In one embodiment, yeast are provided that include a first genetic modification, a second genetic modification, and a third genetic modification. In this embodiment, the first genetic modification reduces expression of a nucleic acid encoding a Gpd1p polypeptide, essentially eliminates expression of a nucleic acid encoding a Gpd1p polypeptide, or results in an absence of a functional Gpd1p polypeptide; the second genetic modification reduces expression of a nucleic acid encoding a Fps1p polypeptide, essentially eliminates expression of a nucleic acid encoding a Fps1p polypeptide, or results in an absence of a functional Fps1p polypeptide; and the third genetic modification results in an increase in the amount of glutamate synthase polypeptide or an increase in the activity of a glutamate synthase polypeptide.

Any of the yeasts disclosed herein can be S. cerevisiae.

Unless otherwise defined, all technical and scientific terms used herein have the same meaning as commonly understood by one of ordinary skill in the art to which this technology belongs. Although methods and materials similar or equivalent to those described herein can be used in the practice or testing of the genetically-engineered yeast described herein, suitable methods and materials are described below. In addition, the materials, methods, and examples are illustrative only and are not intended to be limiting. All publications, patent applications, patents, and other references mentioned herein are incorporated by reference in their entirety.

The details of methods and materials described herein are set forth in the accompanying drawings and the description below. Other features, objects, and advantages will be apparent from the drawings and detailed description, and from the claims.

### DESCRIPTION OF DRAWINGS

FIG. 1 is a restriction map of the construct designated pUC18-RYUR.

FIG. 2 is a restriction map of the construct designated YIplac211-Ppgk1-GLT1.

### DETAILED DESCRIPTION

Physiologically, glycerol plays two roles in yeast. When yeast cells grow anaerobically, excess cytosolic NADH must be re-oxidized to NAD⁺ in the cytosol, which typically occurs via glycerol formation (Van Dijken et al., 1986, FEMS Microbiol. Rev., 32:199-225; Nordstrom, 1968, J. Inst. Brew., 74:429-432). In addition, when yeast cells grow under high osmolarity, glycerol accumulates inside the cell where it acts as an efficient osmolyte that protects the cell against lysis. In commercial-scale fermentations such as those used in ethanol production, however, glycerol is an unwanted by-product that consumes carbon that otherwise would be available in ethanol-producing pathways.

The approaches described herein allow for the metabolic engineering of yeast such that the synthesis and transport of glycerol is disrupted. Due to the disruption in glycerol synthesis, the yeast is further modified to alter the cellular co-factor metabolism of the yeast and maintain the redox balance of the yeast cell. The genetically-engineered yeasts described herein and genetically-engineered yeasts made using the methods described herein typically produce increased amounts of ethanol and reduced amounts of glycerol compared to yeast lacking the corresponding genetic modifications.

Several strategies are provided in this disclosure for disrupting glycerol synthesis and transport in yeast, and for altering the cellular co-factor metabolism in yeast to maintain the redox balance. These strategies are described herein with respect to the gene and polypeptide nomenclature from S. cerevisiae, but the same strategies can be applied to other types of yeast that are or can be used in fermentation reactions, particularly those that are suitable for use in industrial fermentations. Suitable yeasts, in addition to S. cerevisiae include, without limitation, Saccharomyces pastorianus, Pichia stipitis, S. bayanus, and Candida shehatae. The pathways or the gene designations may differ slightly in these other yeasts, but those of skill could readily apply the strategies described herein to modify the corresponding pathways or homologous genes. Genetically engineering yeast is well known to those skilled in the art. See, for example, Jin et al., 2008, Mol. Biol. Cell, 19:284-96.

The ability of yeast to produce glycerol can be disrupted by genetically modifying one of the cytosolic enzymes involved in the synthesis of glycerol. In one example, NAD+-dependent glycerol-3-phosphate dehydrogenase (GPDH), which converts dihydroxyacetone phosphate into glycerol-3-phosphate, can be disrupted. Those of skill, however, understand that, in another example, a phosphatase that converts glycerol-3-phosphate into glycerol (e.g., Gppp) can be disrupted. As used herein, “disruption” of a NAD+-dependent GPDH polypeptide or a phosphatase polypeptide typically refers to a genetic modification that reduces expression of a nucleic acid encoding a NAD+-dependent GPDH or a phosphatase polypeptide; essentially eliminates expression of a nucleic acid encoding a NAD+-dependent GPDH or a phosphatase polypeptide; or results in the absence of a functional NAD+-dependent GPDH or a phosphatase polypeptide. Disrupting a polypeptide involved in the synthesis of glycerol typically causes the accumulation of one or more precursors of glycerol (e.g., dihydroxyacetone phosphate or glycerol-3-phosphate).

In S. cerevisiae, there are two genes designated GPD1 and GPD2 that each encode an active isoenzyme of NAD⁺-dependent GPDH designated Gpdp. Despite the similar physical and catalytic properties of their gene products (Gpd1p and Gpd2p, respectively), the GPD1 and GPD2 genes are differentially regulated at the transcriptional level. Expression of GPD1 is induced by high osmolarity, whereas expression of GPD2 is induced under anaerobic conditions. Consistent with their transcriptional regulation, the enzyme encoded by GPD1 is predominantly responsible for adaptation of S. cerevisiae to high osmolarity, while that encoded by GPD2 is important for maintaining the cellular redox balance under anaerobic conditions. Those of skill in the art would understand that either the GPD1 gene or the GPD2 gene, but typically not both, can be disrupted in S. cerevisiae.

Polypeptides having GPDH activity are assigned to Enzyme Classification (EC) 1.1.1.8 under the IUBMB Enzyme Nomenclature system. Representative GPDH nucleic acid and polypeptide sequences can be found, for example, in GenBank Accession Nos. NC_{—}003424.3; NC_{—}002951.2; NC_{—}009648.1; NT_{—}033779.4; NC_{—}000002.10; NC_{—}006322.1; NC_{—}003281.7; and NC_{—}003279.5. See, also, Baranowski, “α-Glycerophosphate dehydrogenase,” In: Boyer et al., (Eds.), The Enzymes, 2nd Ed., Vol. 7, Academic Press, New York, 1963, pp. 85-96.

The ability of the yeast to produce glycerol also can be disrupted by genetically modifying a polypeptide that transports or helps transport glycerol out of the cell. A polypeptide that transports or helps transport glycerol out of the cell can be, for example, a polyol transporter, a sugar transporter, or specifically a glycerol transporter. In one embodiment, the FPS1 gene from S. cerevisiae, encoding a glycerol permease designated Fps1p, can be disrupted. Typically, at high osmolarity, the Fps1p channel is closed and glycerol is retained inside the cells, where it acts as a compatible solute. After a shift from high to low osmotic strength or upon adaptation to the high osmolarity, the cells generally release the accumulated glycerol to the medium. Those of skill would understand that nucleic acid sequences encoding other glycerol transport polypeptides in S. cerevisiae could be identified and similarly disrupted, as could nucleic acid sequences encoding glycerol transport polypeptides or polypeptides that facilitate glycerol transport in other species or strains of yeast. As indicated herein, “disrupting” a glycerol channel polypeptide typically refers to a genetic modification that reduces expression of a nucleic acid encoding a glycerol channel polypeptide, essentially eliminates expression of a nucleic acid encoding a glycerol channel polypeptide, or results in an absence of a functional glycerol channel polypeptide. Such a disruption generally results in an increase in the accumulation of glycerol in the yeast and also has a down-regulatory effect on glycerol synthesis.

Glycerol transport polypeptides are members of the major intrinsic protein (MIP) family of channel proteins. Among MIPs, two functionally distinct subgroups have been characterized; aquaporins, which allow specific water transfer, and glycerol channels, which are involved in glycerol transport and transport of small neutral solutes. Representative sequences of glycerol transport proteins (also known as glycerol channel polypeptides or facilitators) or variations thereof can be found, for example, in GenBank Accession Nos. NP_{—}013057; NC_{—}001144.4; NC_{—}007946.1; NC_{—}006155.1; NC_{—}010322.1; NC_{—}003143.1; NC_{—}002662.1; and NC_{—}000964.2.

As used herein, a nucleic acid sequence (sometimes referred to as a gene) typically refers to a coding sequence that can be translated into a polypeptide. A nucleic acid sequence also can include regulatory regions (e.g., 5′ or 3′ untranslated region (UTR), promoter sequences, and/or enhancer sequences) associated with the coding sequence. As used herein, nucleic acids (or fragments thereof) include DNA molecules or RNA molecules that contain natural nucleotides and/or nucleotide analogs. Nucleic acids can be single-stranded or double-stranded, and can be circular or linear depending upon the intended use.

A genetic modification that disrupts a polypeptide involved in glycerol synthesis or that disrupts a glycerol transport polypeptide can be in a nucleic acid sequence encoding a polypeptide involved in glycerol synthesis and/or a glycerol transport polypeptide, respectively (e.g., the GPD1, GPD2, and/or FPS1 genes in S. cerevisiae). Alternatively, a genetic modification that disrupts a polypeptide involved in glycerol synthesis or that disrupts a glycerol transport polypeptide can be in a nucleic acid sequence that encodes a polypeptide that, respectively, regulates the expression or function of a polypeptide involved in glycerol synthesis or of a glycerol transport polypeptide.

As used herein, a genetic modification that reduces the expression of a polypeptide involved in glycerol synthesis or of a glycerol transport polypeptide refers to a genetic modification that results in a decrease in the amount of the polypeptide (compared to levels of the polypeptide in wild type yeast) of at least 30% (e.g., at least 40%, 50%, 60%, 70%, 80%, 90%, or 95%). As used herein, a genetic modification that essentially eliminates expression of a polypeptide refers to a genetic modification that results in a decrease in the amount of polypeptide (relative to the amount of polypeptide produced by a wild type yeast) of at least 95% (e.g., 96%, 97%, 98%, 99%, or 100%). As used herein, a genetic modification that results in a decrease in or absence of a functional polypeptide refers to a genetic modification that allows expression of a nucleic acid encoding the polypeptide but that results in a polypeptide that is not able to convert dihydroxyacetone phosphate to glycerol-3-phosphate or glycerol-3-phosphate to glycerol or a polypeptide that is not able to transport glycerol or facilitate transfer of glycerol across the membrane.

A genetic modification as referred to herein can be a substitution or an insertion or deletion of one or more nucleotides. Point mutations include, for example, single nucleotide transitions (purine to purine or pyrimidine to pyrimidine) or transversions (purine to pyrimidine or vice versa) and single- or multiple-nucleotide deletions or insertions. A mutation in a nucleic acid can result in one or more conservative or non-conservative amino acid substitutions in the encoded polypeptide, which may result in conformational changes or loss or partial loss of function, a shift in the reading frame of translation (“frame-shift”) resulting in an entirely different polypeptide encoded from that point on, a premature stop codon resulting in a truncated polypeptide (“truncation”), or a mutation in nucleic acid may not change the encoded polypeptide at all (“silent” or “nonsense”). See, for example, Johnson & Overington, 1993, J. Mol. Biol., 233:716-38; Henikoff & Henikoff, 1992, Proc. Natl. Acad. Sci. USA, 89:10915-19; and U.S. Pat. No. 4,554,101 for disclosure on conservative and non-conservative amino acid substitutions.

Genetic modification can be generated in the nucleic acid of yeast using any number of methods known in the art. For example, site directed mutagenesis can be used to modify nucleic acid sequence. One of the most common methods of site-directed mutagenesis is oligonucleotide-directed mutagenesis. In oligonucleotide-directed mutagenesis, an oligonucleotide encoding the desired change(s) in sequence is annealed to one strand of the DNA of interest and serves as a primer for initiation of DNA synthesis. In this manner, the oligonucleotide containing the sequence change is incorporated into the newly synthesized strand. See, for example, Kunkel, 1985, Proc. Natl. Acad. Sci. USA, 82:488; Kunkel et al., 1987, Meth. Enzymol., 154:367; Lewis & Thompson, 1990, Nucl. Acids Res., 18:3439; Bohnsack, 1996, Meth. Mol. Biol., 57:1; Deng & Nickoloff, 1992, Anal. Biochem., 200:81; and Shimada, 1996, Meth. Mol. Biol., 57:157. Other methods are used routinely in the art to modify the sequence of a polypeptide. For example, nucleic acids containing a genetic modification can be generated using PCR or chemical synthesis, or polypeptides having the desired change in amino acid sequence can be chemically synthesized. See, for example, Bang & Kent, 2005, Proc. Natl. Acad. Sci. USA, 102:5014-9 and references therein.

Since disrupting glycerol synthesis and/or transport of glycerol out of the cell alters the state of redox balance of a cell growing under anaerobic conditions due to an accumulation of NADH, the yeast also can be engineered to effectively reoxidize the excess cytosolic NADH in the absence of glycerol synthesis. In the embodiment shown in the Examples below, excess NADH is effectively reoxidized by over-expressing a nucleic acid sequence encoding a glutamate synthase (GOGAT), which utilizes NADH as a co-factor in the conversion of glutamine to glutamate. It would be understood by those of skill in the art that polypeptides other than GOGAT can be over-expressed or disrupted provided that those polypeptides are involved, either directly or indirectly, in reactions that maintain the cellular redox balance (e.g., by reoxidizing NADH or NADPH). Such polypeptides include, for example, glutamine synthetase (GS) encoded by GLN1, NADP+-dependent glutamate dehydrogenases encoded by GDH1 and GDH3, or a NAD+-dependent glutamate dehydrogenase encoded by GDH2. It would also be understood by those of skill that, rather than over-expressing a nucleic acid, the encoded polypeptide (e.g., GOGAT, GS, NADP+-dependent glutamate dehydrogenase, or NAD+-dependent glutamate dehydrogenase) can be genetically—engineered to exhibit greater activity (compared to a wild type polypeptide) such that the chemical reaction that is facilitated by the genetically-engineered polypeptide takes place at a faster rate relative to the wild type polypeptide. Typically, a balance in a cell's redox potential is reflected by cell growth and sugar consumption.

Polypeptides having glutamate synthase activity are assigned EC 1.4.1.13 under the IUBMB Enzyme Nomenclature system. Representative GLT nucleic acid and polypeptide sequences can be found, for example, in GenBank Accession Nos. NC_{—}003071.4; NC_{—}001136.8; NC_{—}003424.3; NC_{—}007795.1; NC_{—}009077.1; NC_{—}009632.1; and NC_{—}010468.1. See, also, Miller & Stadtman, “Glutamate synthase from Escherichia coli. An iron-sulfide flavoprotein,” J. Biol. Chem., 247:7407-7419, 1972.

There are a number of ways in which a nucleic acid sequence encoding a polypeptide can be over-expressed. For example, the number of copies of a nucleic acid sequence can be increased; a nucleic acid sequence can be genetically engineered so as to be expressed under a different or stronger promoter and/or enhancer; the promoter and/or other regulatory elements of a nucleic acid sequence can be altered so as to direct high levels of expression (e.g., the binding strength of a promoter region for its transcriptional activators can be increased); the half-life of the transcribed mRNA can be increased; the degradation of the mRNA and/or polypeptide can be inhibited; and/or a nucleic acid sequence can be genetically modified as described herein such that the activity of the encoded polypeptide (e.g., rate of conversion, affinity for substrate) is increased. A nucleic acid sequence is considered to be over-expressed if the encoded polypeptide is present at an amount that is at least 20% higher (e.g. at least 25%, 30%, 40%, 50%, 60%, 70%, 80%, 90%, 95%, 99%, 100% higher or more) that will than the amount of polypeptide typically expressed from a corresponding nucleic acid that is not over-expressed. As used herein, “over-expression” also can refer to an increase in activity of a polypeptide (e.g., a polypeptide that has at least two-fold greater activity than a wild type polypeptide).

One or more copies of a nucleic acid sequence to be over-expressed can be present in a construct (also referred to as a vector), or one or more copies of a nucleic acid sequence to be over-expressed can be integrated into the yeast genome. Constructs suitable for over-expressing a nucleic acid are commercially available (e.g., expression vectors) or can be produced by recombinant DNA technology methods routine in the art. See, for example, Akada et al. (2002, Yeast, 19:17-28; and Mitchell et al. (1993, Yeast, 9:715-22). In addition, methods for stably integrating nucleic acid into the yeast genome are known and routine in the art. See, for example, Methods in Enzymology: Guide to Yeast Genetics and Molecular Biology, Vol. 194, 2004, Abelson et al., eds., Academic Press.

A construct containing a nucleic acid sequence can have elements necessary for expression operably linked to such a nucleic acid sequence, and further can include sequences such as those encoding a selectable marker (e.g., an antibiotic resistance gene), and/or those that can be used in purification of a polypeptide (e.g., 6×His tag). A construct also can include one or more origins of replication. Elements necessary for expression include nucleic acid sequences that direct and regulate expression of nucleic acid coding sequences. One example of an element necessary for expression is a promoter sequence. Representative promoters include, without limitation, the promoter from the phosphoglycerate kinase (PGK) gene, the promoter from the triose phosphate isomerase (TPI1) gene and the promoter from the alcohol dehydrogenase (ADH1) gene. Elements necessary for expression also can include intronic sequences, enhancer sequences, response elements, or inducible elements that modulate expression of a nucleic acid coding sequence.

Elements necessary for expression can be of bacterial, yeast, insect, plant, mammalian, fungal, or viral origin, and vectors or constructs can contain a combination of elements from different origins. Elements necessary for expression are described, for example, in Goeddel, 1990, Gene Expression Technology: Methods in Enzymology, 185, Academic Press, San Diego, Calif. As used herein, operably linked means that a promoter and/or other regulatory element(s) are positioned in a construct relative to a nucleic acid sequence encoding a GOGAT polypeptide in such a way as to direct or regulate expression of the nucleic acid sequence. In certain instances, the nucleic acid sequences and/or the elements necessary for expression may be codon optimized to obtain optimal expression in yeast. See, for example, Bennetzen & Hall, 1982, J. Biol. Chem., 257:3026-31.

Nucleic acid sequences (e.g., expression vectors) can be introduced into yeast cells or other host cells using any of a number of different methods. Such methods include, without limitation, electroporation, calcium phosphate precipitation, heat shock, lipofection, microinjection, lithium chloride, lithium acetate, z-mercaptoethanol, and viral-mediated nucleic acid transfer. “Host cells” can include, in addition to yeast cells, cells that can be used in standard molecular biology techniques to manipulate and produce the nucleic acids and polypeptides described herein. “Host cells” include, without limitation, bacterial cells (e.g., E. coli), insect cells, plant cells or mammalian cells (e.g., CHO or COS cells). “Yeast cells,” including the genetically-engineered yeast cells described herein, and other types of “host cells” refers, not only to the particular cell(s) into which a nucleic acid sequence was introduced, but also to the progeny of such cells.

In addition to disrupting the ability of yeast to produce glycerol and/or transport glycerol out of the cell as described herein and modifying the yeast to maintain the redox balance of the yeast cell as described herein, one or more additional nucleic acid sequences can be genetically modified. Such additional nucleic acids can be associated with glycerol synthesis, glycerol metabolism, cofactor metabolism, or ethanol tolerance, or can be associated with, for example, growth characteristics on different medium or at different temperatures. The expression of such additional nucleic acids can be disrupted as described herein or over-expressed as described herein.

### Nucleic Acids and Polypeptides

As used herein, an “isolated” nucleic acid molecule (represented by a nucleic acid sequence) is a nucleic acid molecule that is separated from other nucleic acid molecules that are usually associated with the reference nucleic acid molecule in the genome. Thus, an “isolated” nucleic acid molecule includes, without limitation, a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a construct (e.g., a cloning vector, or an expression vector) for convenience of manipulation or to express a fusion polypeptide. In addition, an isolated nucleic acid molecule can include an engineered nucleic acid molecule such as a recombinant or a synthetic nucleic acid molecule.

Nucleic acids can be obtained using techniques routine in the art. For example, isolated nucleic acids can be obtained using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid molecule. Isolated nucleic acids also can be chemically synthesized, either as a single nucleic acid molecule or as a series of oligonucleotides. In addition, isolated nucleic acids also can be obtained by mutagenesis.

Amplification of nucleic acids can be used to produce or detect a nucleic acid. Conditions for amplification of a nucleic acid and detection of an amplification product are known to those of skill in the art (see, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188). Modifications to the original PCR also have been developed. For example, anchor PCR, RACE PCR, or ligation chain reaction (LCR) are additional PCR methods known in the art (see, e.g., Landegran et al., 1988, Science, 241:1077 1080; and Nakazawa et al., 1994, Proc. Natl. Acad. Sci. USA, 91:360 364).

Hybridization of nucleic acids also can be used to obtain or detect a nucleic acid. Hybridization between nucleic acid molecules is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). For oligonucleotide probes less than about 100 nucleotides, Sambrook et al. discloses suitable Southern blot conditions in Sections 11.45-11.46. The Tm between a sequence that is less than 100 nucleotides in length and a second sequence can be calculated using the formula provided in Section 11.46. Sambrook et al. additionally discloses prehybridization and hybridization conditions for a Southern blot that uses oligonucleotide probes greater than about 100 nucleotides (see Sections 9.47-9.52). Hybridizations with an oligonucleotide greater than 100 nucleotides generally are performed 15-25° C. below the Tm. The Tm between a sequence greater than 100 nucleotides in length and a second sequence can be calculated using the formula provided in Sections 9.50-9.51 of Sambrook et al. Additionally, Sambrook et al. recommends the conditions indicated in Section 9.54 for washing a Southern blot that has been probed with an oligonucleotide greater than about 100 nucleotides.

The conditions under which membranes containing nucleic acids are prehybridized and hybridized, as well as the conditions under which membranes containing nucleic acids are washed to remove excess and non-specifically bound probe can play a significant role in the stringency of the hybridization. Such hybridizations and washes can be performed, where appropriate, under moderate or high stringency conditions. Such conditions are described, for example, in Sambrook et al. section 11.45-11.46. For example, washing conditions can be made more stringent by decreasing the salt concentration in the wash solutions and/or by increasing the temperature at which the washes are performed. In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium.

It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane. A nucleic acid molecule is deemed to hybridize to a target nucleic acid but not to a non-target nucleic acid if hybridization to a target nucleic acid is at least 5-fold (e.g., at least 6-fold, 7-fold, 8-fold, 9-fold, 10-fold, 20-fold, 50-fold, or 100-fold) greater than hybridization to a non-target nucleic acid. The amount of hybridization can be quantitated directly on a membrane or from an autoradiograph using, for example, a PhosphorImager or a Densitometer (Molecular Dynamics, Sunnyvale, Calif.).

The term “purified” polypeptide (or protein) as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide always would be considered “purified.”

Polypeptides can be purified from natural sources (e.g., a biological sample) by known methods such as DEAE ion exchange, gel filtration, and hydroxyapatite chromatography. A purified polypeptide also can be obtained, for example, by expressing a nucleic acid molecule in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis. As described elsewhere in this disclosure, polypeptides can be produced using recombinant expression vectors or constructs.

Antibodies can be used to detect the presence or absence of polypeptides. Techniques for detecting polypeptides using antibodies include enzyme linked immunosorbent assays (ELISAs), Western blots, immunoprecipitations and immunofluorescence. An antibody can be polyclonal or monoclonal, and usually is detectably labeled. An antibody having specific binding affinity for a polypeptide can be generated using methods well known in the art. The antibody can be attached to a solid support such as a microtiter plate using methods known in the art (see, for example, Leahy et al., 1992, BioTechniques, 13:738-743). In the presence of an appropriate polypeptide, an antibody-polypeptide complex is formed.

Detection of an amplification product, a hybridization complex, or a polypeptide-antibody complex usually is accomplished using detectable labels. The term “labeled” with regard to an agent (e.g., an oligonucleotide, a polypeptide, or an antibody) is intended to encompass direct labeling of the agent by coupling (i.e., physically linking) a detectable substance to the agent, as well as indirect labeling of the agent by reactivity with another reagent that is directly labeled with a detectable substance. Detectable substances include various enzymes, prosthetic groups, fluorescent materials, chemoluminescent materials, bioluminescent materials, and radioactive materials.

### Methods of Using Yeast Strains

The genetically-engineered yeast described herein or genetically-engineered yeast made using the methods described herein can be used in fermentation reactions to metabolize carbohydrates and produce ethanol or another alcohol. A genetically-modified yeast as described herein produces little to no glycerol. Therefore, genetically-modified yeast as described herein produces higher amounts of ethanol than yeast that do not have the corresponding genetic modifications. The genetically-engineered yeast described herein can produce ethanol at levels that are increased by up to about 3% or more (e.g., about 1.0%, 1.2%, 1.5%, 1.8%, 2.0%, 2.3%, 2.6%, 2.9%, 3.0%, 3.1%, or 3.2%) compared to yeast lacking the corresponding genetic modifications. In addition, the genetically-engineered yeast described herein can produce at least 35% (e.g., at least 40%, 45%, 50%, 60%, or more) less glycerol than does yeast lacking the corresponding genetic modifications.

The preferred growth conditions (e.g., temperature, pH, agitation, and/or oxygenation) for yeast genetically-modified as described herein can be determined using routine experimentation. In certain instances, the genetically-modified yeast described herein exhibit osmotolerance (e.g., withstands up to 35% sugar concentration) and an alcohol tolerance of at least about 15% (at 38° C.).

In accordance with the present disclosure, there may be employed conventional molecular biology, microbiology, biochemical, and recombinant DNA techniques within the skill of the art. Such techniques are explained fully in the literature. Certain methods and materials are further described in the following examples, which do not limit the scope of the claims.

### EXAMPLES

### Example 1

### Cultivation Conditions

Yeast strains were cultivated at 30° C. on 2% agar plates or in liquid culture with rich YP medium containing 1% yeast extract, 2% Bacto-peptone, 2% glucose) or with minimal YNB medium containing 0.67% yeast nitrogen base without amino acids. 20 μg/ml of uracil was added to minimal medium to satisfy auxotrophic requirements or withheld to select for transformants. Escherichia coli TOP10 F′ was used to propagate plasmids. Escherichia coli cells were cultured in Luria-Bertani medium (1% bacto tryptone, 0.5% bacto yeast extract, 1% NaCl) and transformed to ampicillin resistance by standard methods. Yeast transformations were performed by the lithium acetate method.

### Example 2

### Fermentation Conditions

Microaerobic batch fermentation was carried out at 30-37° C. in 200 ml in-house-manufactured bioreactors sealed with screw caps or in 500 ml shake flasks sealed with parafilm. The working volume for both fermenter was 150 ml. The composition of the fermentation medium was corn mash containing 20-30% reducing sugar supplemented with 0.02% K2HPO4, 0.02% MgSO4, 0.05% (NH4)2HPO4, 0.05% urea. An overnight preculture prepared in rich YP medium was inoculated into the fermenter to reach an initial OD660 1.5-2.0.

### Example 3

### Plasmid and Strain Construction

All primers used for construction of plasmids and strains are listed in Table 1.

TABLE 1

| Primer | Primer | Sequence |
|---|---|---|
| function | name | (restriction site underlined) |
| Primers for | Rep1-U | 5′-GGG CCC GGA TCC GAG CAG CAT |
| plasmid | | AAA CGA CTG CT-3′ (BamH I) |
| pUC18-RYUR | | (SEQ ID NO:1) |
| construction | Rep1-D | 5′-GGG CCC TCT AGA ACG CTC AAT |
| | | GTT GTT CAT GA-3′ (Xbal I) |
| | | (SEQ ID NO:2) |
| | Rep2-U | 5′-GGG CCC GTC GAC GAG CAG CAT |
| | | AAA CGA CTG CT-3′ (Sal I) |
| | | (SEQ ID NO:3) |
| | Rep2-D | 5′-GGG CCC CTG CAG ACG CTC AAT |
| | | GTT GTT CAT GA-3′ (Pst I) |
| | | (SEQ ID NO:4) |
| | URA3-U | 5′-GGG CCC TCT AGA GTA GTC TAG |
| | | TAC CTC CTG TG-3′ (XbaI) |
| | | (SEQ ID NO:5) |
| | URA3-D | 5′-GGG CCC GTC GAC GAA AAG TGC |
| | | CAC CTG ACG TC-3′ (Sal I) |
| | | (SEQ ID NO:6) |
| Primers for | GLT1 | 5′-GGG CCC GGT ACC TTT CTG AGC |
| plasmid | prom-U | ACT GTC AGG AG-3′ (KpnI) |
| YIp1ac211- | | (SEQ ID NO:7) |
| _{PGK1}-GL T1 | GLT1 | 5′-GGG CCC GGA TCC TGA TTT CAA |
| construction | prom-D | CAC TGG CAT GC-3′ (BamH I) |
| | | (SEQ ID NO:8) |
| | GLT1-U | 5′-GGG CCC GTC GAC ATG CCA GTG |
| | | TTG AAA TCA GA-3′ (Sal I) |
| | | (SEQ ID NO:9) |
| | GLT1- | 5′-GGG CCC CTG CAG TTT TAG TAT |
| | D: | CGA CCA TTT CA-3′ (Pst I) |
| | | (SEQ ID NO:10) |
| | PGK1 | 5′-GGG CCC GGA TCC AGG CAT TTG |
| | prom-U | CAA GAA TTA CTC-3′ (BamH I) |
| | | (SEQ ID NO:11) |
| | PGK1 | 5′-GGG CCC GTC GAC TGT TTT ATA |
| | prom-D | TTT GTT GTA AAA AGT AG-3′ |
| | | (Sal I) |
| | | (SEQ ID NO:12) |
| Primers for | KGPD1- | 5′-CAC ATT CCA AAG GAT TTC AGA |
| GPD1 | U | GGC GAG GGC AAG GAC GTC GAC |
| deletion | | GAC GTT GTA AAA CGA CG-3′ |
| | | (SEQ ID NO:13) |
| | KGPD1- | 5′-AGT GGG GGA AAG TAT GAT ATG |
| | D | TTA TCT TTC TCC AAT AAA TGG |
| | | AAA CAG CTA TGA CCA TG-3′ |
| | | (SEQ ID NO:14) |
| Primers for | KGPD2- | 5′-CTC TTT CCC TTT CCT TTT CCT |
| GPD2 | U | TCG CTC CCC TTC CTT ATC AAC |
| deletion | | GAC GTT GTA AAA CGA CG-3′ |
| | | (SEQ ID NO:15) |
| | KGPD2- | 5′-GCA ACA GGA AAG ATC AGA GGG |
| | D | GGA GGG GGG GGG AGA GTG TGG |
| | | AAA CAG CTA TGA CCA TG-3′ |
| | | (SEQ ID NO:16) |
| Primers for | KFPS1- | 5′-TCA ACA AAG TAT AAC GCC TAT |
| FPS1 | U | TGT CCC AAT AAG CGT CGGTAC GAC |
| deletion | | GTT GTA AAA CGA CG-3′ |
| | | (SEQ ID NO:17) |
| | KFPS1- | 5′-CAT CAT GTA TAG TAG GTG ACC |
| | D | AGG CTG AGT TCA TGT CAA CGG |
| | | AAA CAG CTA TGA CCA TG-3′ |
| | | (SEQ ID NO:18) |

### Example 4

### Construction of a Selectable Marker-Recoverable Gene Knockout Cassette

For multi-round gene manipulation, we need to make a URA3 based gene knockout cassette, in which, the URA3 gene can be used repeatedly as a selectable marker for multiple gene manipulation. To this end, plasmid pUC18-RYUR was constructed

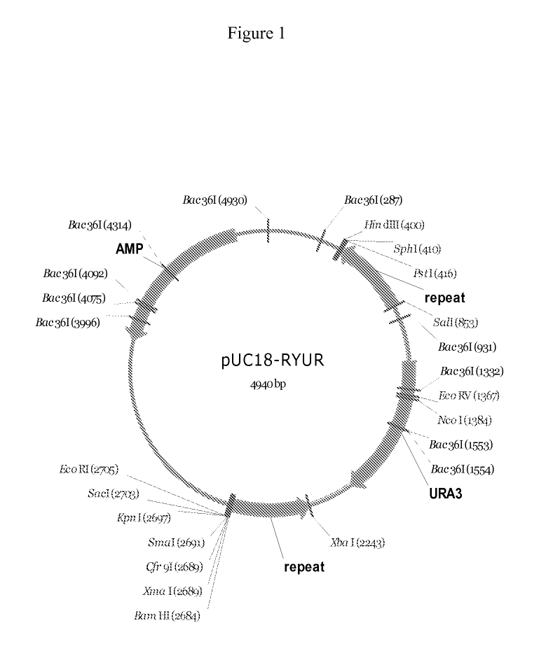

First, a 435 by DNA fragment corresponding to nucleotide sequence 4165652 by to 4166066 of B. subtilis 168 genome were PCR amplified with primers Rep1-U and Rep1-D flanked by the restriction sites BamHI and XbaI, respectively. The resulting PCR product was digested by BamHI and XbaI and then ligated with the same enzyme pair digested pUC18, resulting in plasmid pUC18-R; Second, the yeast URA3 gene was PCR amplified from YEplac195 with primers URA3-U, corresponding to the vector sequence 1940 to 1959 flanked by restriction site XbaI and URA3-D corresponding to the vector sequence 3323 to 3304 flanked by restriction site Sail, respectively. The resulting PCR product was digested by XbaI and SalI and then ligated with the same enzyme pair digested pUC18-R, resulting in plasmid pUC18-RYU; Finally, the exact same DNA sequence of B. subtilis 168 genome as described above was PCR amplified with primers Rep2-U and Rep2-D flanked by restriction sites SalI and PstI, respectively. The resulting PCR fragment was digested by SalI and PstI and then ligated with the same enzyme pair digested plasmid pUC18-RYU, creating plasmid pUC18-RYUR (FIG. 1).

### Example 5

### Deletion of FPS1

To delete FPS1, plasmid pUC18-RYUR was PCR amplified with primers KFPS1-U and KFPS1-D. KFPS1-U contains, at its 3′ portion, sequences corresponding to pUC18 sequences 371 to 389 and, at its 5′ portion, sequences corresponding to positions −100 to −61 with respect to the ATG start codon of the FPS1 gene; KFPS1-D contains, at its 3′ portion, sequences corresponding to pUC18 sequences 479 to 461 and, at its 5′ portion, sequences corresponding to positions 2250 to 2211 with respect to the ATG start codon of the FPS1 gene. This PCR product was then used to transform yeast. Transformants were isolated on minimal medium lacking uracil and checked by diagnostic PCR for the correct integration of the RYUR cassette. The isolates, in which the targeted gene deletion had occurred, were subjected onto FOA plates to select for loop-out of the URA3 gene through homologous recombination between the repeat sequences flanking the URA3 gene in the deletion cassette.

### Example 6

### Deletion of GPD1

To delete GPD1, plasmid pUC18-RYUR was PCR amplified with primers KGPD1-U and KGPD1-D. KGPD1-U contains, at its 3′ portion, sequences corresponding to pUC18 sequences 371 to 389 and, at its 5′ portion, sequences corresponding to positions 601 to 640 with respect to the ATG start codon of the GPD1 gene; KGPD1-D contains, at its 3′ portion, sequences corresponding to pUC18 sequences 479 to 461 and, at its 5′ portion, sequences corresponding to positions 1216 to 1177 with respect to the ATG start codon of the GPD1 gene. This PCR product was then used to create GPD1 deletion strain as described above for deletion of FPS1.

### Example 7

### Deletion of GPD2

For deletion of GPD2, plasmid pUC18-RYUR was PCR amplified with primers KGPD2-U and KGPD2-D. KGPD2-U contains, at its 3′ portion, sequences corresponding to pUC18 sequences 371 to 389 and, at its 5′ portion, sequences corresponding to positions −40 to −1 with respect to the ATG start codon of the GPD2 gene; KGPD2-D contains, at its 3′ portion, sequences corresponding to pUC18 sequences 479 to 461 and, at its 5′ portion, sequences corresponding to positions 1363 to 1324 with respect to the ATG start codon of the GPD2 gene. This PCR product was then used to create GPD2 deletion strain as described above for deletion of FPS1.

### Example 8

### Over-Expression of GLT1

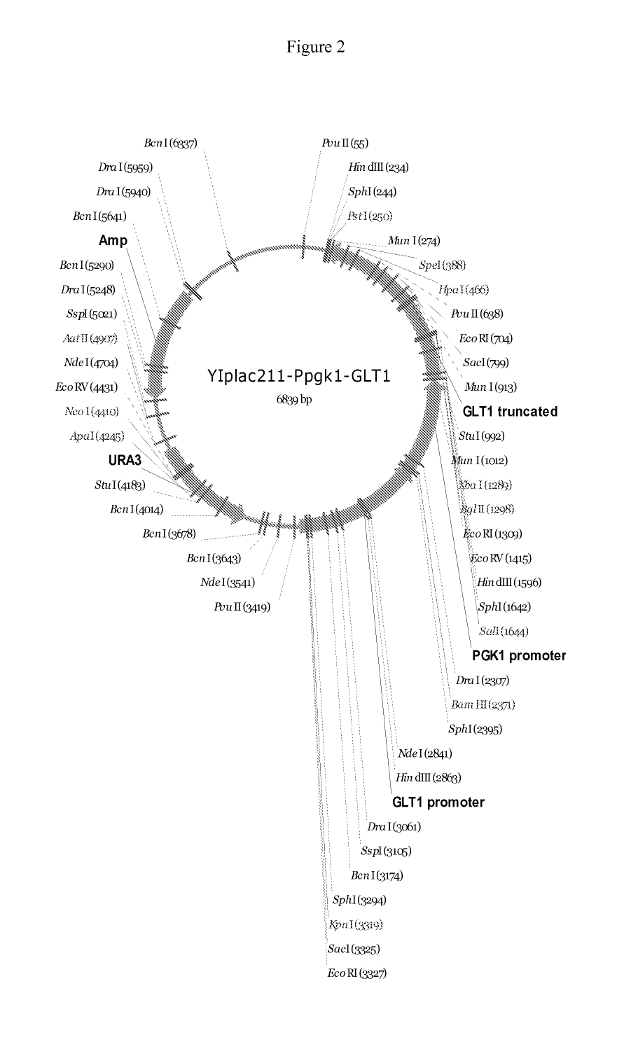

For GLT1 over-expression, plasmid YIplac211-Ppgk1-GLT1 that harbors 5′ portion of the GLT1 ORF fused to the PGK1 promoter and, upstream of the PGK1 promoter, a DNA fragment corresponding to positions 18 to −920 with respect to the ATG start codon of the GLT1 gene, was constructed as follows: (1) the first 1390 by GLT1 ORF was PCR amplified with primers GLT1-U corresponding to position 1 to 20 with respect to the ATG start codon of the GLT1 gene, flanked by the restriction site SalI, and GLT1-D corresponding to position 1390 to 1371 with respect to the ATG start codon of the GLT1 gene flanked by the restriction site PstI, respectively. The resulting PCR product was digested by SalI and PstI and then ligated with the same enzyme pair digested YIplac211, resulting in plasmid YIplac211-GLT1t; (2) Primers GLT1prom-U corresponding to position −920 to −901 with respect to the ATG start codon of the GLT1 gene flanked by the restriction site KpnI, and GLT1prom-D corresponding to position 18 to −2 with respect to the ATG start codon of the GLT1 gene flanked by the restriction site BamHI were used to amplify a DNA fragment upstream of the GLT1 ORF. This PCR product was digested by KpnI and BamHI and then ligated with the same enzyme pair digested YIplac211-GLT1t, creating plasmid YIplac211-GLT1p-GLT1t. (3) Primers PGK1prom-U corresponding to position −701 to −721 with respect to the ATG start codon of the PGK1 gene flanked by the restriction site BamHI, and PGK1prom-D corresponding to position −1 to −26 with respect to the ATG start codon of the PGK1 gene flanked by the restriction site SalI were used to amplify a DNA fragment upstream of the PGK1 ORF that contains the promoter of the gene. This PCR product was digested by BamHI and SalI and ligated with same enzyme pair digested YIplac211-GLT1p-GLT1, and the resulting plasmid was designated YIplac211-Ppgk1-GLT1 (FIG. 2).

To replace GLT1 promoter with the PGK1 promoter in the genome, YIplac211-Ppgk1-GLT1 was digested by BglII and the linearized plasmid was used for yeast transformation. Isolation and verification of the transformants and subsequent loop-out of the vector sequence, including the URA3 gene, were performed essentially as described above.

To evaluate the genetically-engineered yeast described herein, yeast cultures were grown at 30° C. in corn mash containing 25% reducing sugar. Biomass (OD 600 nm), remaining reducing sugar, glycerol and ethanol were measured at 48 h. The FTG2 strain produced about 3% more ethanol and at least 35% less glycerol compared to the unmodified strain. The results of those experiments are shown in Table 2.

TABLE 2
Fermentation performance of yeast strains

| | | Reducing | | |
|---|---|---|---|---|
| | OD | sugar | Glycerol | Ethanol |
| Strains | 600 nm | (g/100 ml) | (g/100 ml) | (g/100 ml) |
| YC-DM (unmodified) | 33.34 | 0.73 | 0.95 | 12.10 |
| gpd2Δ fps1Δ PGK1-GLT1 | 29.21 | 1.29 | 0.52 | 12.56 |
| fps1Δ PGK1-GLT1 | 32.15 | 0.95 | 0.75 | 12.25 |
| gpd1Δ PGK1-GLT1 | 26.86 | 1.27 | 0.73 | 12.19 |
| gpd2Δ PGK1-GLT1 | 29.07 | 1.05 | 0.61 | 12.28 |
| gpd1Δ fps1Δ PGK1-GLT1 | 25.20 | 1.13 | 0.66 | 12.27 |

### Other Embodiments

Only a few implementations are disclosed. However, it is understood that variations and enhancements of the described implementations and other implementations can be made based on what is described and illustrated in this document.

1. A yeast comprising a first genetic modification, a second genetic modification, and a third genetic modification,
wherein the first genetic modification disrupts a polypeptide involved in the synthesis of glycerol;
wherein the second genetic modification disrupts a polypeptide that transports or helps transport glycerol out of the cell; and
wherein the third genetic modification increases the amount of a polypeptide that maintains the redox balance in the cell.

2. A yeast comprising a first genetic modification, a second genetic modification, and a third genetic modification,
wherein said first genetic modification reduces expression of a nucleic acid encoding a GPDH polypeptide, essentially eliminates expression of a nucleic acid encoding a GPDH polypeptide, or results in an absence of a functional GPDH polypeptide, thereby disrupting glycerol synthesis and resulting in an accumulation of one or more precursors of glycerol;
wherein said second genetic modification reduces expression of a nucleic acid encoding a glycerol channel polypeptide, essentially eliminates expression of a nucleic acid encoding a glycerol channel polypeptide, or results in an absence of a functional glycerol channel polypeptide, thereby resulting in an accumulation of glycerol in the yeast; and
wherein said third genetic modification increases the amount of a polypeptide that reoxidizes NADH.

3. A S. cerevisiae yeast comprising a first genetic modification, a second genetic modification, and a third genetic modification,
wherein said first genetic modification reduces expression of a nucleic acid encoding a Gpd1p or Gpd2p polypeptide, essentially eliminates expression of a nucleic acid encoding a Gpd1p or Gpd2p polypeptide, or results in an absence of a functional Gpd1p or Gpd2p polypeptide;
wherein said second genetic modification reduces expression of a nucleic acid encoding a Fps1p polypeptide, essentially eliminates expression of a nucleic acid encoding a Fps1p polypeptide, or results in an absence of a functional Fps1p polypeptide; and
wherein said third genetic modification results in an increase in the amount of glutamate synthase polypeptide or an increase in the activity of a glutamate synthase polypeptide.

4. The yeast of claim 1 or 2, wherein said yeast is S. cerevisiae.

5. The yeast of any of claims 1 to 3, wherein first or second genetic modification is a genetically-engineered point mutation, deletion, or insertion.

6. The yeast of any of claims 1 to 3, wherein said first or second genetic modification reduces expression of said polypeptide by at least 30%.

7. The yeast of any of claims 1 to 3, wherein said third genetic modification is the presence of a strong promoter operably linked to a nucleic acid encoding said polypeptide.

8. The yeast of any of claims 1 to 3, wherein said yeast produces reduced amounts of glycerol and increased amounts of ethanol compared to a yeast lacking a corresponding first, second and/or third genetic modifications.

9. The yeast of any of claims 1 to 3, wherein said yeast produces up to about 3% more ethanol than a yeast lacking a corresponding first, second and/or third genetic modifications.

10. The yeast of any of claims 1 to 3, further comprising one or more additional genetic modifications.

11. A method of fermenting, comprising contacting biomass with the yeast of any of claims 1 to 3.

12. A method of making a yeast, comprising
introducing a first genetic modification into the yeast, wherein the first genetic modification is in a nucleic acid that encodes a polypeptide involved in the synthesis of glycerol;
introducing a second genetic modification into the yeast, wherein the second genetic modification is in a nucleic acid that encodes a polypeptide that transports or helps transport glycerol out of the cell; and
introducing a third genetic modification into the yeast, wherein the third genetic modification increases the amount of a polypeptide that maintains the redox balance of the yeast cells.

13. The method of claim 12, wherein said first genetic modification is in a nucleic acid that encodes a GPDH polypeptide, wherein said second genetic modification is in a nucleic acid that encodes a glycerol channel polypeptide, and wherein said third genetic modification results in over-expression of a polypeptide that reoxidizes NADH.

14. The method of claim 12 or 13, wherein the yeast produces less glycerol and more ethanol than a corresponding yeast lacking the first, second and third genetic modifications.

15. A yeast comprising a first genetic modification, a second genetic modification, and a third genetic modification, wherein said first genetic modification essentially eliminates expression of a nucleic acid encoding a Gpd2p polypeptide; wherein said second genetic modification essentially eliminates expression of a nucleic acid encoding a Fps1p polypeptide; and wherein said third genetic modification results in an increase in the amount of a glutamate synthase polypeptide.

16. The yeast of claim 15, wherein said yeast is a strain designated FTG2.

17-19. (canceled)

20. A yeast comprising a first genetic modification, a second genetic modification, and a third genetic modification,
wherein said first genetic modification reduces expression of a nucleic acid encoding a Gpd1p polypeptide, essentially eliminates expression of a nucleic acid encoding a Gpd1p polypeptide, or results in an absence of a functional Gpd1p polypeptide;
wherein said second genetic modification reduces expression of a nucleic acid encoding a Fps1p polypeptide, essentially eliminates expression of a nucleic acid encoding a Fps1p polypeptide, or results in an absence of a functional Fps1p polypeptide; and
wherein said third genetic modification results in an increase in the amount of glutamate synthase polypeptide or an increase in the activity of a glutamate synthase polypeptide.

21. The yeast of claim 20, wherein said yeast is S. cerevisiae.

22. A S. cerevisiae yeast comprising a first genetic modification, a second genetic modification, and a third genetic modification,
wherein said first genetic modification reduces expression of a nucleic acid encoding a NAD+-dependent glycerol-3-phosphate dehydrogenase (GPDH) polypeptide, essentially eliminates expression of a nucleic acid encoding a GPDH polypeptide, or results in an absence of a functional GPDH polypeptide;
wherein said second genetic modification reduces expression of a nucleic acid encoding a Fps1p polypeptide, essentially eliminates expression of a nucleic acid encoding a Fps1p polypeptide, or results in an absence of a functional Fps1p polypeptide; and
wherein said third genetic modification results in an increase in the amount of a NADP+- or NAD+-dependent glutamate dehydrogenase polypeptide or an increase in the activity of a NADP+- or NAD+-dependent glutamate dehydrogenase polypeptide.

23. The yeast of claim 22, wherein said GDPH is selected from the group consisting of Gpdp1 or Gpdp2.

24. The yeast of claim 22, wherein said NADP+-dependent glutamate dehydrogenase polypeptide is encoded by one or more nucleic acids selected from the group consisting of GDH1 and GDH3.

25. The yeast of claim 22, wherein said NAD+-dependent glutamate dehydrogenase polypeptide is encoded by a GDH2 nucleic acid.

26. A S. cerevisiae yeast comprising a first genetic modification, a second genetic modification, and a third genetic modification,
wherein said first genetic modification reduces expression of a nucleic acid encoding a phosphatase polypeptide that converts glycerol-3-phosphate into glycerol, essentially eliminates expression of a nucleic acid encoding a phosphatase polypeptide that converts glycerol-3-phosphate into glycerol, or results in an absence of a functional phosphatase polypeptide that converts glycerol-3-phosphate into glycerol;
wherein said second genetic modification reduces expression of a nucleic acid encoding a Fps1p polypeptide, essentially eliminates expression of a nucleic acid encoding a Fps1p polypeptide, or results in an absence of a functional Fps1p polypeptide; and
wherein said third genetic modification results in an increase in the amount of glutamate synthase polypeptide or an increase in the activity of a glutamate synthase polypeptide.

27. The yeast of claim 26, wherein said phosphatase polypeptide that converts glycerol-3-phosphate into glycerol is Gppp.

28. A S. cerevisiae yeast comprising a first genetic modification, a second genetic modification, and a third genetic modification,
wherein said first genetic modification reduces expression of a nucleic acid encoding a phosphatase polypeptide that converts glycerol-3-phosphate into glycerol, essentially eliminates expression of a nucleic acid encoding a phosphatase polypeptide that converts glycerol-3-phosphate into glycerol, or results in an absence of a functional phosphatase polypeptide that converts glycerol-3-phosphate into glycerol;
wherein said second genetic modification reduces expression of a nucleic acid encoding a Fps1p polypeptide, essentially eliminates expression of a nucleic acid encoding a Fps1p polypeptide, or results in an absence of a functional Fps1p polypeptide; and
wherein said third genetic modification results in an increase in the amount of a NADP+- or NAD+-dependent glutamate dehydrogenase or an increase in the activity of a NADP+- or NAD+-dependent glutamate dehydrogenase.

29. The yeast of claim 28, wherein said phosphatase polypeptide that converts glycerol-3-phosphate into glycerol is Gppp.

30. The yeast of claim 28, wherein said NADP+-dependent glutamate dehydrogenase is encoded by one or more nucleic acids selected from the group consisting of GDH1 and GDH3.

31. The yeast of claim 28, wherein said NAD+-dependent glutamate dehydrogenase is encoded by a GDH2 nucleic acid.
